(19) United States
(12) Patent Application Publication
(10) Pub. No.: US 2005/0273009 A1
(43) Pub. Date: Dec. 8, 2005

(54) Method and apparatus for co-display of inverse mode ultrasound images and histogram information

(76) Inventors: Harald Deischinger, Frankenmarkt (AT); Helmut Brandl, Pfaffing (AT)

Correspondence Address: DEAN D. SMALL;C/O ARMSTRONG TEASDALE LLP, ONE METROPOLITAN SQUARE, SUITE 2600, ST. LOUIS, MO, 63102-2740, US

(21) Appl. No.: 10/858,880

(22) Filed: Jun. 2, 2004

(51) Int. Cl. A61B 8/00

(52) U.S. Cl. 600/437; 600/456

(57) ABSTRACT

An ultrasound system is provided for analyzing a region of interest. The ultrasound system includes a probe for acquiring ultrasound information associated with the region of interest and a memory for storing a volumetric data set corresponding to at least a subset of the ultrasound information for at least a portion of the region of interest. The system further includes at least one processor for generating histogram information based on the volumetric data set and for generating ultrasound images based on the volumetric data set. The processor formats the histogram information and the ultrasound images to be co-displayed. The system further includes a display for simultaneously co-displaying the histogram information and the ultrasound images.

### BACKGROUND OF THE INVENTION

The present invention generally related to an ultrasound method and apparatus for analyzing a region of interest and more particularly to a method and apparatus for co-displaying inverse mode ultrasound images and histogram information.

Ultrasound systems have long existed for analyzing various regions of interest, such as in medical applications and in non-medical fields. Conventional ultrasound systems display the ultrasound information in a variety of formats and configurations. By way of example, existing ultrasound systems may display a series of two dimensional images or slices based on a volume of acquired data where the position of each slice is determined by the user. Along with the set of two dimensional slices or images, a rendered image (e.g. a three dimensional representation) may be separately or simultaneously displayed with one or more of the two dimensional images or slices. Conventional systems provide the user with various functionality to rotate the images and adjust the parameters used to generate the images. The displayed images present the ultrasound information in various manners, such as gray scale levels representative of the intensity of echo signals received from each scan of the region of interest, as well as color information, inverse gray levels and the like.

Conventional systems also offer modes in which non-image based information is presented to the user, such as statistical measurements of particular physiologic parameters, graphs, bar charts and the like.

However, conventional systems have been unable to combine images and certain types of non-image information in an easily viewable and adjustable manner.

### BRIEF DESCRIPTION OF THE INVENTION

An ultrasound system is provided for analyzing a region of interest. The ultrasound system includes a probe for acquiring ultrasound information associated with the region of interest and a memory for storing a volumetric data set corresponding to at least a subset of the ultrasound information for at least a portion of the region of interest. The system further includes at least one processor for generating histogram information based on the volumetric data set and for generating an ultrasound image based on the volumetric data set. The processor formats the histogram information and the ultrasound image to be co-displayed. The system further includes a display for simultaneously co-displaying the histogram information and the ultrasound image.

Optionally, the ultrasound image may comprise a collection of images that includes at least one of a volume rendered image and a set of orthogonal image slices, one or more of which are co-displayed with the histogram information. Optionally, the ultrasound images and/or the histogram information may be generated based upon inverse levels of gray scale values stored within voxels defining the volumetric data set. Optionally, the display may present the ultrasound images and the histogram information in separate first and second windows that at least partially overlap one another, with the positions of each window being adjustable by the user with click and drag functions of a mouse.

The system may further comprise an inverse map memory that stores an invert function. The processor may then calculate inverted data values based on the invert function and the volumetric data set. At least one of the histogram information and the ultrasound image may be representative of the inverted data values.

Optionally, the system may include a user interface configured to receive a threshold parameter. The processor may update histogram information and the ultrasound images in real-time based on user adjustment of the threshold parameter.

In accordance with at least one alternative embodiment, a method is provided for analyzing a region of interest. A method includes acquiring ultrasound information associated with the region of interest and storing a volumetric data set corresponding to at least a subset of the ultrasound information for at least a portion of the region of interest. The method further comprises generating histogram information based on the volumetric data set and generating an ultrasound image based on the volumetric data set. The method also includes formatting the histogram information and the ultrasound image to be co-displayed and then simultaneously co-displaying the histogram information and the ultrasound image.

### BRIEF DESCRIPTION OF THE DRAWINGS

FIG. 1 illustrates a block diagram of an ultrasound system formed in accordance with one embodiment of the present invention.

FIG. 2 illustrates a block diagram of an ultrasound system formed in accordance with an alternative embodiment of the present invention.

FIG. 3 illustrates a block diagram of an ultrasound system formed in accordance with an alternative embodiment of the present invention.

FIG. 4 illustrates a block diagram of an ultrasound system formed in accordance with an alternative embodiment of the present invention.

FIG. 5 illustrates a method setting forth steps carried out in accordance with at least one embodiment of the present invention.

FIG. 6 illustrates a screen shot in which ultrasound images and histogram information are co-displayed simultaneously in accordance with one embodiment of the present invention.

FIG. 7 illustrates an inverse map utilized in accordance with certain embodiments of present invention.

FIG. 8 illustrates a surface rendering map utilized in accordance with certain embodiments of the present invention.

### DETAILED DESCRIPTION OF THE INVENTION

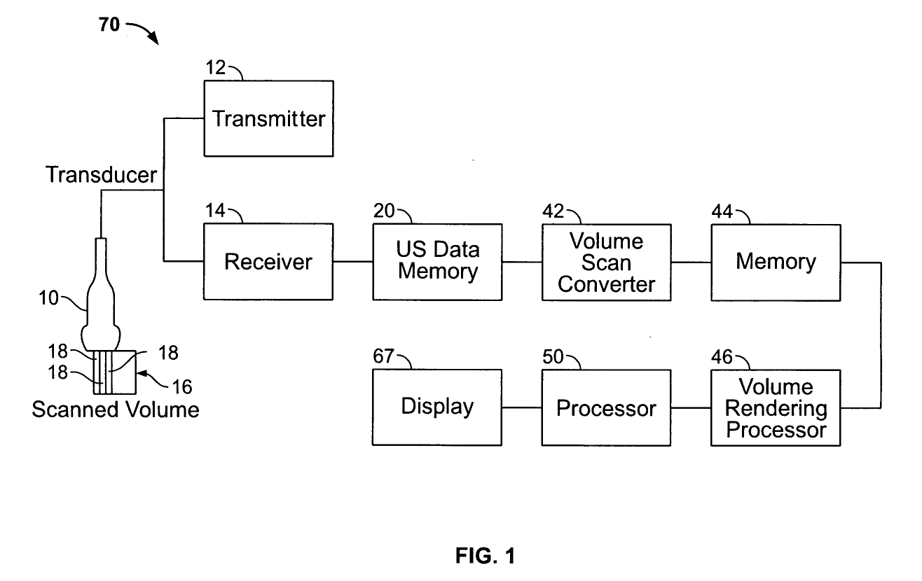

FIG. 1 illustrates an ultrasound system 70 formed in accordance with one embodiment of the present invention. The system 70 includes a probe 10 connected to a transmitter 12 and a receiver 14. The probe 10 transmits ultrasonic pulses and receives echoes from structures inside of a scanned ultrasound volume 16. Memory 20 stores ultrasound data from the receiver 14 derived from the scanned ultrasound volume 16. The volume 16 may be obtained by various techniques (e.g., 3D scanning, real-time 3D scanning, 2D scanning with transducers having positioning sensors, freehand scanning using a voxel correlation technique, 1.25D, 1.5D, 1.75D, 2D or matrix array transducers and the like).

The probe 10 is moved, such as along a linear or arcuate path, or electronically steered when using a 2D array, while scanning a region of interest (ROI). At each linear or arcuate position, the transducer 10 obtains scan planes 18. The scan planes 18 are stored in the memory 20, and then passed to a volume scan converter 42. In some embodiments, the probe 10 may obtain lines instead of the scan planes 18, and the memory 20 may store individual or subsets of lines obtained by the probe 10 rather than the scan planes 18. The volume scan converter 20 may store lines obtained by the transducer 10 rather than the scan planes 18. The volume scan converter 42 creates data slices from the US data memory 20. The data slices are stored in slice memory 44 and are accessed by a volume rendering processor 46. The volume rendering processor 46 performs volume rendering upon the data slices. The output of the volume rendering processor 46 is passed to the processor 50 and display 67.

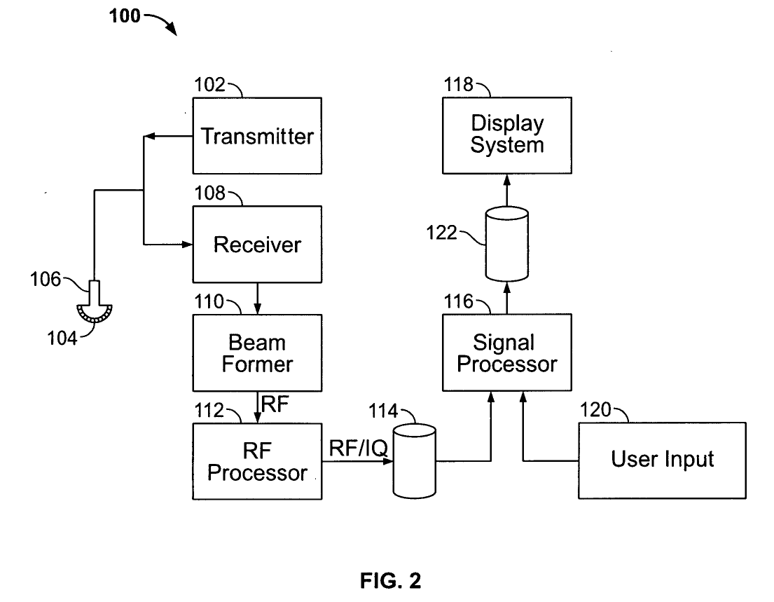

FIG. 2 illustrates a block diagram of an ultrasound system 100 formed in accordance with an embodiment of the present invention. The ultrasound system 100 includes a transmitter 102 which drives transducers 104 within a probe 106 to emit pulsed ultrasonic signals into a body. A variety of geometries may be used. The ultrasonic signals are back-scattered from structures in the body, like blood cells or muscular tissue, to produce echoes which return to the transducers 104. The echoes are received by a receiver 108. The received echoes are passed through a beamformer 110, which performs beamforming and outputs an RF signal. The RF signal then passes through an RF processor 112. Alternatively, the RF processor 112 may include a complex demodulator (not shown) that demodulates the RF signal to form IQ data pairs representative of the echo signals. The RF or IQ signal data may then be routed directly to RF/IQ buffer 114 for temporary storage. A user input 120 may be used to input patient data, scan parameters, a change of scan mode, and the like.

The ultrasound system 100 also includes a signal processor 116 to process the acquired ultrasound information (i.e., RF signal data or IQ data pairs) and prepare frames of ultrasound information for display on display system 118. The signal processor 116 is adapted to perform one or more processing operations according to a plurality of selectable ultrasound modalities on the acquired ultrasound information. Acquired ultrasound information may be processed in real-time during a scanning session as the echo signals are received. Additionally or alternatively, the ultrasound information may be stored temporarily in RF/IQ buffer 114 during a scanning session and processed in less than real-time in a live or off-line operation.

The ultrasound system 100 may continuously acquire ultrasound information at a frame rate that exceeds 50 frames per second—the approximate perception rate of the human eye. The acquired ultrasound information is displayed on the display system 118 at a slower frame-rate. An image buffer 122 is included for storing processed frames of acquired ultrasound information that are not scheduled to be displayed immediately. Preferably, the image buffer 122 is of sufficient capacity to store at least several seconds worth of frames of ultrasound information. The frames of ultrasound information are stored in a manner to facilitate retrieval thereof according to its order or time of acquisition. The image buffer 122 may comprise any known data storage medium.

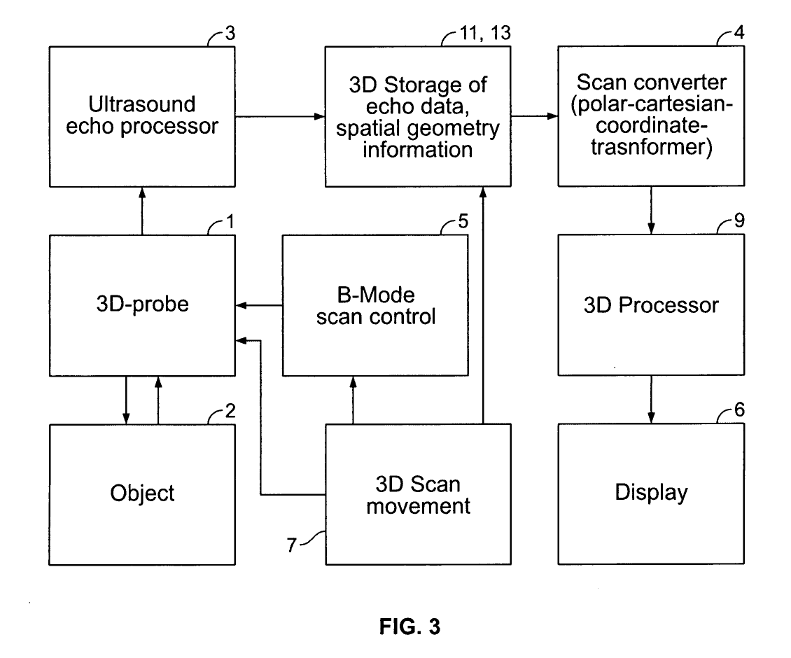

FIG. 3 illustrates a system for the continuous volume scanning of an object by the means of ultrasound waves. The system includes an ultrasound-echo-processor 3, polar cartesian-coordinate transformer (“Scanconverter”) 4, B-mode scan-control 5 and display 6. The system also includes a 3D or volume scanning probe 1, controller for the volume scan movement 7, control-unit for B-mode scanning, 3D-processor 9, 3D-storage of echo data 11 and a unit to store spatial geometry information 13.

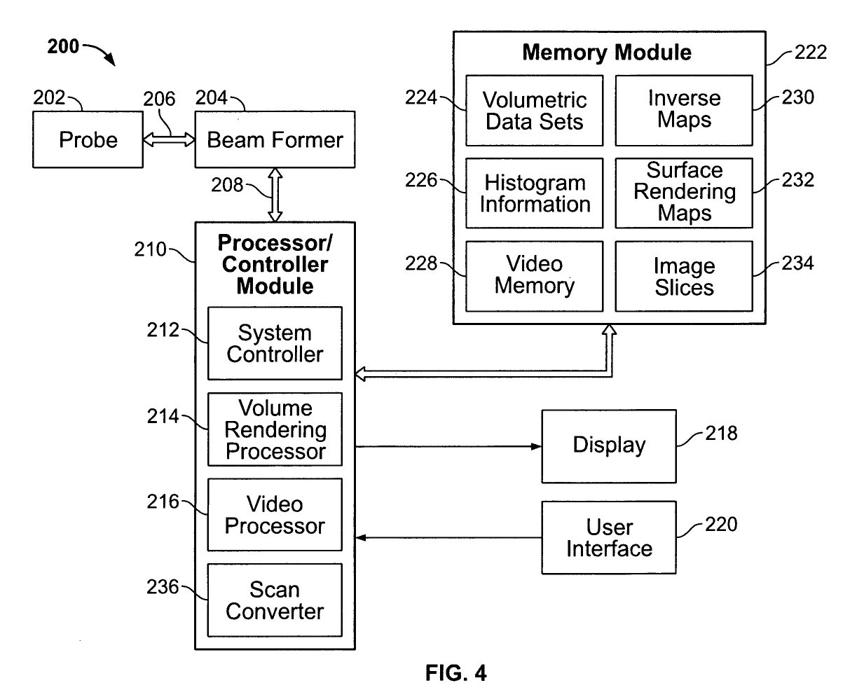

FIG. 4 illustrates an ultrasound system 200 formed in accordance with an alternative embodiment of the present invention.

The ultrasound system 200 includes a probe 202 which communicates with a beamformer 204 over a transmit/receive link 206. The transmit/receive link 206 conveys transmit information to the probe 204 and conveys received echo-data from the probe 202 to the beamformer 204. The beamformer 204 is connected at link 208 to a processor/controller module 210 which comprises one or more controllers and processors. The module 210 may comprise a single processor (such as in a personal computer and the like) which performs all processing operations explained throughout the present application. Alternatively, the module 210 may include multiple processors arranged to carry out multi-processing in a shared manner. Alternatively, the module 210 may represent a hardware implemented configuration of individual boards provided in a cage where each board includes dedicated processors and memory and related components associated with the various functions of the ultrasound system 200.

In the example of FIG. 4, the module 210 includes and performs the functionality of a system controller 212, a volume rendering processor 214 and a video processor 216. The volume rendering processor 214 performs, at least, volume rendering operations to generate rendered images based upon stored ultrasound data for one or more volumes. The video processor 216 controls formatting, writing to and reading from one or more video memory buffers to control the information presented on the display 218. The system controller 212 coordinates and controls operation of at least processors 214 and 216. A user interface 220 is provided to permit the user to enter various types of information. The user interface 220 may include a keyboard, a mouse, a track ball and the like.

The ultrasound system 200 also includes a memory module 222 that is denoted in FIG. 4 as a common block. Optionally, one or more separate memory sections may be utilized in connection with each of the various types of stored information. For example, the memory module 222 may include a personal computer hard drive, a remote data base interconnected to the ultrasound system 200 over the internet or some other networking link. Optionally, the memory module 222 may include various buffers, cash memory, RAM, ROM and the like, distributed within the ultrasound system 200 on various boards, chips and the like. The memory module 222 includes common or separate memory space for storing volumetric data sets 224, histogram information 226, video memory 228, invert maps 230, surface rendering maps 232 and image slices 234.

The volumetric data sets 224 comprise one or more sets of ultrasound data representative of a volume within the region of interest. Successive volumetric data sets 224 may be stored in separate memories, such as scan converter memories or alternatively in a common FIFO type buffer in which each new successive volume is acquired and pushed into the front end of the buffer, while the oldest volumetric data set within the buffer is being processed and/or read out. Each volumetric data set comprises a three dimensional array of voxels, each voxel of which contains a gray scale value associated with a particular point in object space within the region of interest. Optionally, the voxels may store not only gray scale values, but also information related to motion within the corresponding object space (e.g. a Doppler value).

The histogram information 226 includes one or more parameters utilized when analyzing the gray scale values of the voxels within a volumetric data set 224. By way of example, the parameters may include high and low threshold parameters selected and adjustable by the user denoting cutoff points in grayscale value intensity. The histogram information 226 also contains the results of a histogram analysis of a corresponding volumetric data set 224. Histograms include a count of the member of voxels at each gray level. The low threshold parameter is user adjustable along the range of potential gray levels.

For example, when a user selects a desired low threshold parameter and a corresponding volumetric data set 224 is analyzed, the histogram information 226 may count the number of voxels above and below the threshold parameters. Based on the number of voxels above and below the threshold various subvolumes within the volumetric data set 224 may also be calculated since each voxel is of equal and known size. By way of example only, if a voxel is a 0.5 millimeter cube, by counting the number of voxels above and below the threshold, the volumes of the region of interest above and below the threshold are determined.

The invert maps 230 stored in memory module 222 may include one or more maps representing function(s) utilized by the processor/control module 210 to generate inverted gray scale or level intensity values.

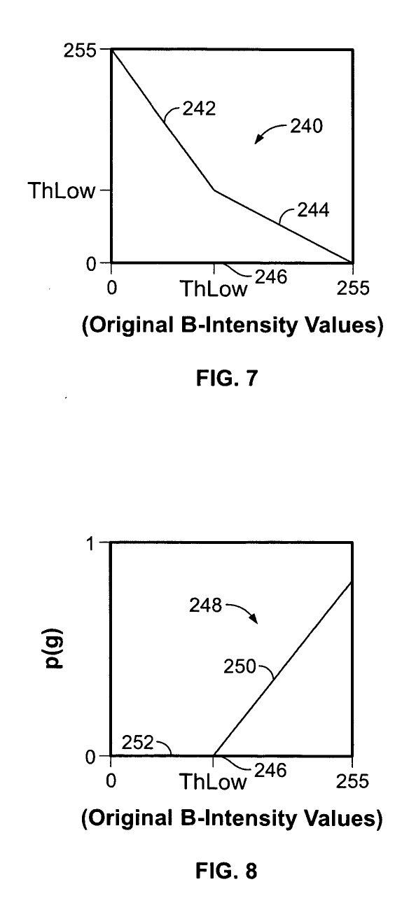

FIG. 7 illustrates a graph of an exemplary inverse function 240 where the horizontal axis of the graph represents the input gray scale and the vertical axis represents the output gray scale. The invert function 240 is a non-linear function, having first and second sections 242 and 244. In the example of FIG. 7, sections 242 and 244 are both linear, but have different slopes and intersect at the threshold parameter 246. Section 242 has a steeper negative slope than that of section 244. Alternatively, sections 242 and 244 may be defined by a common or different non-linear functions. The invert function 240 is used by the volume rendering processor 214 to produce invert rendered images from gray scale values in the accessed volumetric data set 224.

Returning to FIG. 4, the memory module 222 further includes one or more surface rendering maps 232 that are utilized by the volume rendering processor 214 to construct a rendered volume that is subsequently displayed by display 218.

FIG. 8 illustrates a graph of an exemplary surface rendering function 248. The horizontal axis of the graph represents the input gray scale, while the vertical axis represents the output opacity value. The surface rendering function 242 also includes a complex structure with sections 250 and 252 having different slopes and intersecting at the threshold parameter 246. The threshold parameter 246 in FIG. 8 represents the same threshold parameter as illustrated in FIG. 7 that defined the intersection between sections 242 and 244 of the inverse map 240. The threshold parameter 246 is adjustable by the user in real-time, in that as the user adjusts the threshold parameter, new images and histogram information are presented shortly thereafter (e.g. in less than 0.25 to 5 sec). The term real-time as used throughout is intended to indicate that ultrasound images or histogram information is displayed to the user in a sufficiently short period of time after the user adjusts the threshold parameter, that the user considers it to be real-time (e.g. in less than 0.25 to 5 sec).

Returning to FIG. 4, the memory module 222 also stores image slices 234 which are produced by the volume scan converter 236 based upon selections by the user, via the user interface 220. For example, the user may identify, through the user interface 220, the position of desired planes along which image slices are desired. With this information, the volume scan converter 236 operates upon a corresponding volumetric data set 224 to generate the image slices. When generating the image slices, the volume scan converter 236 may produce inverted images (e.g., images comprised of gray levels inverted based on the invert function 240) such as to generate A-plane, B-plane, C-plane images and the like. It is also possible that the image slices are presented with the original gray scales where values below the threshold 246 are marked in color. (e.g. pink)

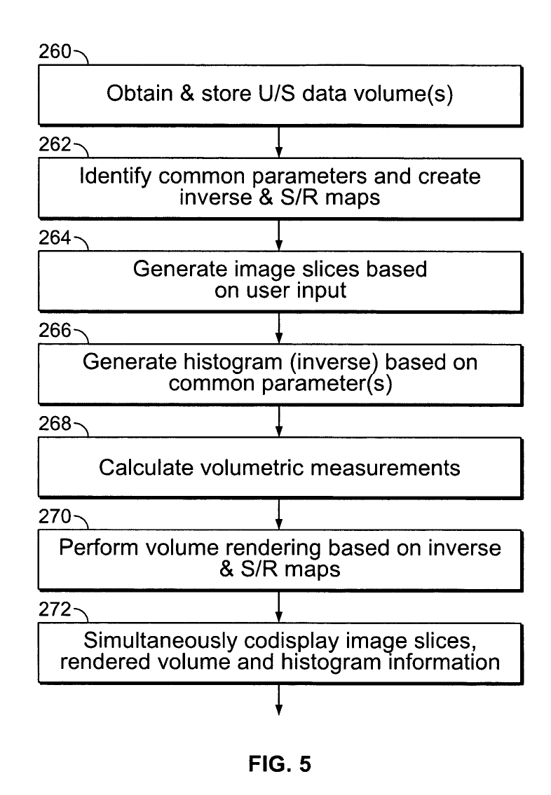

FIG. 5 illustrates a processing sequence carried out in accordance with an embodiment of the present invention. In FIG. 5, at step 260, ultrasound data is obtained and stored in one or more volumetric data sets in the memory module 222. At step 262, a common parameter, such as the threshold parameter 246, is identified and used to create an invert map 230 and a surface rendering map 232. With reference to FIGS. 7 and 8, once the threshold parameter 246 is identified, at step 262, the invert function 240 and the surface rendering functions 248 are generated by the processor 214.

At step 264, image slices 234 are generated based on a user input, such as identifying a particular point or series of locations in the volumetric data set 224. The image slices 234 may be orthogonal to one another, but need not necessarily be orthogonal. Examples of image slices include the A plane, the B plane, the C plane, the I plane and the like.

At step 266, a histogram is generated and stored in the histogram information 226. The histogram maybe generated based on a volumetric data set 224.

At step 268, the histogram is analyzed to calculate volume related histogram information. At step 270, the volume rendering processor 214 performs a volume rendering operation based on the invert and surface rendering maps 230 and 232 and on a corresponding volumetric data set 224. At step 272, the image slices 234, rendered image and histogram information are simultaneously co-displayed under control of the video processor 216 by the display 218.

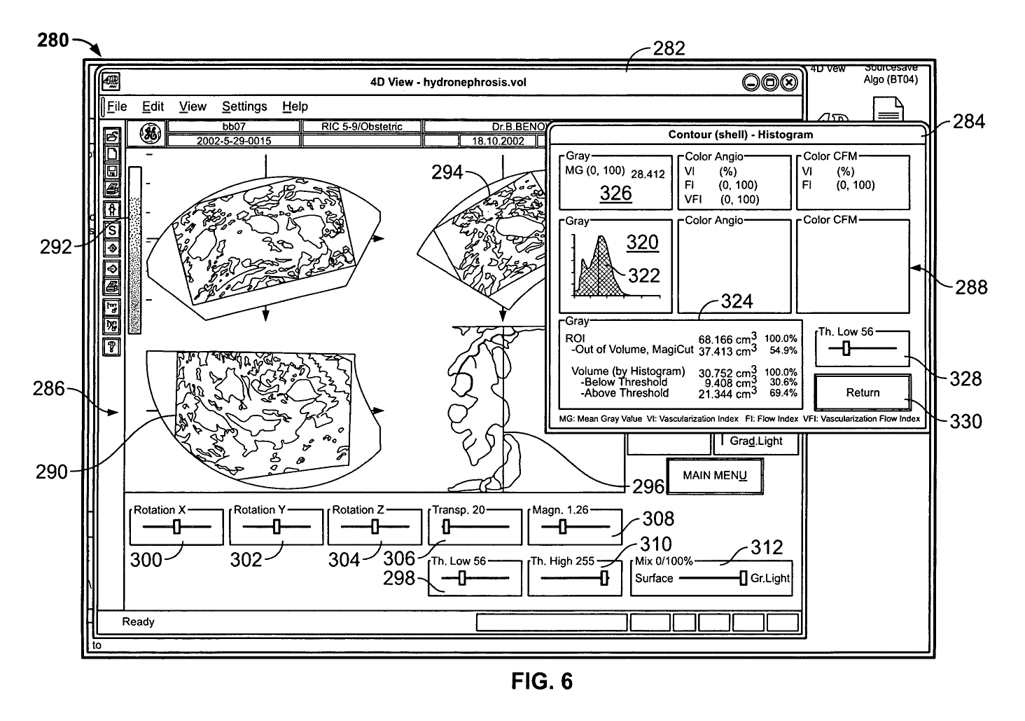

FIG. 6 illustrates a screen shot 280 of the information that is co-displayed simultaneously on the display 218 to the user. The screen shot 280 includes windows 282 and 284 that overlap one another and may be moved by the user using a click and drag function of a trackball or mouse. While the window 284 overlaps in front of window 282, they may be reversed when the user simply clicks on window 282. Each window 282 and 284 may be adjusted in size by the user through the mouse by grabbing a boarder of the corresponding window 282 and 284 and dragging it a desired distance. Window 282 includes ultrasound images generally denoted at reference numeral 286, while window 284 generally illustrates histogram information denoted by reference numeral 288. The ultrasound images 286 include a set of image slices 290, 292 and 294 which, in the example of FIG. 6, correspond to orthogonal image planes (e.g. the A plane, B plane and C plane). The ultrasound images 286 also include a rendered image 296 which in the example of FIG. 6 constitutes an invert rendered image in that each gray level of the underlying volumetric data set 224 has been converted based upon a corresponding invert map 230 prior to generation of the surface rendered image 296.

The window 282 also includes multiple adjustable parameters including a threshold parameter bar 298 that is graphically illustrated as a bar that may be grabbed and pulled utilizing the mouse and/or a track ball. As the threshold parameter bar 298 is adjusted between left-most and right most extremes, the value of the threshold parameter 246 is similarly adjusted. The value of the threshold parameter 246 is also identified (in the example of FIG. 6 it is denoted as “56”).

The window 282 include other adjustment sliders or bars, such an X-rotation bar 300, Y-rotation bar 302, Z-rotation bar 304, transparency bar 306, magnification bar 308, high threshold parameter bar 310 and surface mix bar 312. As the user adjust one or more of the parameters denoted by bars 298-312, the ultrasound images 286 and the histogram information 288 are updated in real-time (e.g. in less than 0.25 to 5 sec).

Turning to the histogram information 288, a graph 320 is presented where the horizontal axis denotes each discrete gray scale intensity and the vertical axis denotes the number of counts at each intensity within the corresponding volumetric data set 224. The graph 320 includes a threshold marker 322 identifying the gray scale value associated with the low threshold tab 298. The histogram information 288 also includes a series of gray scale statistics 324, such as the volume in cubic centimeters 1) of the region of interest, 2) of the “out of volume” area, 3) of the “in volume” area, 4) the “in volume” area below the threshold and 5) the “in volume” area above the threshold. The “out of volume” area represents a section of the volumetric data set 224 that the user has identified to be removed from the subsequent histogram analysis and thus is not reflected in the graph 320.

As the threshold parameter bar 298 is adjusted, the corresponding threshold parameter 246 is adjusted and the appropriate processor within the processor/controller module 210 adjusts both of the inverse function 240 and the surface rendering function 248. Once the inverse function 240 and surface rendering function 248 are adjusted, subsequent image slices 234 or rendered images are generated based on the updated functions and thus reflect changes in how gray level values are mapped. Also, the appropriate processor within the processor/controller module 210, performs subsequent histogram calculations based on the updated inverse and surface rendered functions 240 and 248. The histogram information 288 and ultrasound images 286 generated based on the adjusted threshold parameter 246 are displayed immediately upon generation. Hence, the user views, in real time (e.g., less than 0.25 to 5 sec.) the results of changing the threshold parameter 246 in the ultrasound images 286 and histogram information 288.

The histogram information 288 also includes the mean gray value 326, the vascular index (VI) the flow index (FI), and the vascularzation flow index (VFI) for various modes, such as color angio and color CFM. The window 284 also includes a threshold parameter bar 328 which performs the same function as the threshold parameter bar 298 in window 282. Offering the same threshold parameter bar 328 and 298 on different windows permits the user added ease in adjusting the parameter. A return button 330 is included in window 284. The user selects the return tab 330 when it is desired to switch to a different window (e.g. window 282).

In accordance with the forgoing, method and apparatus are provided which permit the user to invert a volumetric data set 224 before performing a volume rendering operation. The volume rendering operation may constitute surface rendering, surface rendering utilizing gradient light, surface rendering with depth shading, maximum intensity projection (MIP), minimum intensity projection, and the like. When the image slices are displayed, they may be displayed with invert intensities and they may be shown in color to further highlight regions having very low gray scale levels.

When the user desires to remove a section of the volume from the statistical analysis, (otherwise known as “MagiCut”), the user selects the section to be removed prior to the volume rendering and histogram calculation operations.

While the invention has been described in terms of various specific embodiments, those skilled in the art will recognize that the invention can be practiced with modification within the spirit and scope of the claims.

1. An ultrasound system, comprising:
a probe acquiring ultrasound information associated with a region of interest;
memory storing a volumetric data set corresponding to at least a subset of said ultrasound information for at least a portion of the region of interest;
a processor generating histogram information based on said volumetric data set and generating an ultrasound image based on said volumetric data set, said processor formatting said histogram information and said ultrasound image to be co-displayed; and
a display simultaneously co-displaying said histogram information and said ultrasound image.

2. The ultrasound system of claim 1, wherein said processor generates at least one of a volume rendered image and a set of orthogonal image slices as said ultrasound image to be co-displayed with said histogram information.

3. The ultrasound system of claim 1, wherein said volumetric data set comprises voxels of gray-scale values, said processor generating said ultrasound image based on inverted values of said gray-scale values.

4. The ultrasound system of claim 1, wherein said volumetric data set comprises voxels of gray-scale values, said processor generating said histogram based on inverted values of said gray-scale values.

5. The ultrasound system of claim 1, wherein said volumetric data set comprises voxels of gray-scale values, said histogram information and said ultrasound image representing inverted values of said gray-scale values.

6. The ultrasound system of claim 1, wherein said display presents said ultrasound image and said histogram information in first and second windows.

7. The ultrasound system of claim 1, wherein said display presents said ultrasound image and said histogram information in first and second windows that at least partially overlap one another.

8. The ultrasound system of claim 1, further comprising invert map memory storing an invert function, said processor calculating inverted data values based on said invert function and said volumetric data set, at least one of said histogram information and said ultrasound image being representative of said invert data values.

9. The ultrasound system of claim 1, further comprising an user interface configured to receive a threshold parameter, said processor updating said histogram information and said ultrasound image in real-time based on user adjustment of said threshold parameter.

10. The ultrasound system of claim 1, further comprising memory storing a threshold parameter, said processor counting an amount of said volumetric data set above and below said threshold parameter to generate said histogram information.

11. The ultrasound system of claim 1, further comprising memory storing a threshold parameter, said processor shading pixels in said ultrasound image with one of first and second gray-scale levels depending on whether corresponding data values in said volumetric data set are above/below said threshold parameter.

12. A method for analyzing a region of interest, comprising:
acquiring ultrasound information associated with the region of interest;
storing a volumetric data set corresponding to at least a subset of said ultrasound information for at least a portion of the region of interest;
generating histogram information based on said volumetric data set;
generating an ultrasound image based on said volumetric data set;
formatting said histogram information and said ultrasound image to be co-displayed; and
simultaneously co-displaying said histogram information and said ultrasound image.

13. The method of claim 12, wherein said generating an ultrasound image further comprises generating at least one of a volume rendered image and a set of orthogonal image slices as said ultrasound image to be co-displayed with said histogram information.

14. The method of claim 12, wherein said volumetric data set comprises voxels of gray-scale values, said generating an ultrasound image further comprising generating said ultrasound image based on invert values of said gray-scale values.

15. The method of claim 12, wherein said volumetric data set comprises voxels of gray-scale values, said generating an ultrasound image further comprising generating said histogram based on invert values of said gray-scale values.

16. The method of claim 12, wherein said volumetric data set comprises voxels of gray-scale values, said histogram information and said ultrasound image representing invert of said gray-scale values.

17. The method of claim 12, said displaying including presenting said ultrasound image and said histogram information in first and second windows.

18. The method of claim 12, said displaying including presenting said ultrasound image and said histogram information in first and second windows that at least partially overlap one another.

19. The method of claim 12, further comprising storing an invert function and calculating invert data values based on said invert function and said volumetric data set, at least one of said histogram information and said ultrasound image being representative of said invert data values.

20. The method of claim 12, further comprising receiving a threshold parameter and updating said histogram information and said ultrasound image in real-time based on adjustment of said threshold parameter.

21. The method of claim 12, further comprising storing a threshold parameter and counting an amount of said volumetric data set above and below said threshold parameter to generate said histogram information.

22. The method of claim 12, further comprising storing a threshold parameter and shading pixels in said ultrasound image with one of first and second gray-scale levels depending on whether corresponding data values in said volumetric data set are above/below said threshold parameter.

23. The method of claim 12, further comprising generating volume information regarding the region of interest based on a number of voxels above and below said threshold parameter and a predetermined size of each voxel, said histogram information including said volume information.
